(19) United States
(12) Patent Application Publication
(10) Pub. No.: US 2009/0124908 A1
(43) Pub. Date: May 14, 2009

(54) Method And Apparatus For Detecting Ultrasound Contrast Agents In Arterioles

(75) Inventors: Patrick G. Rafter, Windham, NH (US); George A. Brock-Fisher, Andover, MA (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/916,611

(22) PCT Filed: Jun. 2, 2006
(86) PCT No.: PCT/IB2006/051774

§ 371 Date: Jul. 2, 2008

Related U.S. Application Data
(60) Provisional application No. 60/687,845, filed on Jun. 6, 2005.

(51) Int. Cl. A61B 8/00

(52) U.S. Cl. 600/458

(57) ABSTRACT

A method and apparatus for ultrasound imaging of microbubbles of a contrast agent in arterioles while all microbubbles of the contrast agent have been eliminated in the capillaries of a patient and tissue signal response to ultrasound imaging is suppressed. This method and apparatus permits ultrasound imaging for detecting coronary artery disease without the need for a stress test.

The present invention relates to a method and apparatus for detecting ultrasound contrast agents in arterioles. In particular, the invention relates to diagnosing coronary artery disease without the need for a stress test by detecting the presence of ultrasound contrast agents microbubble in larger vessels including the arterioles.

Ultrasound Contrast agents act as intravascular tracers and are approved for various uses throughout the world. In the US, FDA has approved the use of contrast for left ventricular opacification to aid in the delineation of endocardial borders in echo studies. In Europe, there are radiology indications as well including enhancement of the macro and microvasculature. However a great deal of clinical research is ongoing for other uses of contrast agents. Myocardial Contrast Echo (MCE), the ability to assess perfusion of the myocardium with echo, is one of the hottest areas of research in echo. The first FDA approval for assessment of MCE is expected to occur in 2006 with others to follow.

In order to assess coronary artery disease (CAD) a patient typically has to undergo some form of stress test. This is due to the heart's ability to compensate for a stenosis (partial blockage) in one of the main coronary arteries to maintain resting coronary blood flow. Compensation occurs by dilation of arterioles to account for the pressure drop across the stenosis. This helps maintain capillary pressure and this is critical for maintaining perfusion. However, after about an 85-90% stenosis the body has exhausted its coronary flow reserve (i.e., its ability to dilate arterioles). For increasing stenosis above 85-90% resting blood flow begins to decrease. In order to diagnose patients with CAD and non-flow limiting stenosis at rest some form of stress test is given—either with ECG, Echo or nuclear perfusion. These tests involve a patient running on a treadmill to obtain a higher heart rate or the use of an inotropic drug (i.e., Dobutamine) or a vasodilator. All of these tests are time consuming and carry some risk and discomfort for the patient.

During the systolic portion of a cardiac cycle the contraction of the heart squeezes blood forward in the venules and backwards in the arterioles. If the blood volume of the arterioles is increased—such as in the case of a coronary stenosis, there is more blood to be squeezed from these vessels. The velocity of this blood will be much higher than that in the capillaries. This allows for the possibility to isolate the arterioles based on velocity differences during systole. Since these vessels are too small to obtain a Doppler signal in the presence of a very strong tissue signal other methods have to be used. One such method uses microbubbles to enhance the signal from blood. Also, since microbubbles can be destroyed with ultrasound this means that destruction could be used to isolate signals from arterioles.

Using ultrasound at an energy high enough to destroy the microbubbles in an imaging plane and imaging with a frame rate such that the microbubbles in the arterioles have enough time to flow back into the scan plane can isolate the microbubbles in the arterioles. At these destructive power levels and frame rates of greater than 1-2 Hz or so, microbubbles don't have enough time to reach the capillaries. However in order to make this possible, imaging techniques have to be developed that are sensitive to small number of microbubbles while suppressing tissue signals at MI's that are destructive (i.e. MI's>=0.2). Techniques based purely on harmonics often have poor tissue suppression due to the presence of tissue harmonic signal at the powers required to destroy microbubbles. Therefore the tissue signal will mask the signal from contrast agents with these techniques. Techniques to image arterioles based on imaging in between the harmonics (i.e., ultraharmonics) or with power doppler techniques were disclosed in U.S. Pat. No. 6,730,036. These techniques are effective in reducing tissue signal at these destructive MI's but suffer from insufficient signal/noise at the power levels (typically MI>0.2 and <0.8 depending on the contrast agent and patient) and frame rates (typically frame rate >=5 and <=25 Hz) required to work effectively. At even higher power levels, the signal to noise ratio of these techniques increases but since a “thicker slice” of contrast in the myocardium is destroyed a lower frame rate is required to allow a sufficient number of microbubbles to replenish the imaging plane or subvolume even in the arterioles. Forcing these techniques to work at slower frame rates allows more time for arterioles to refill but also makes it harder to isolate arteriole signals from capillary signals since capillaries also have more time to refill. Alternatively, the higher MI could be used at a higher frame rate but would require large doses of contrast agent to improve signal to noise leading to attenuation of much of the myocardium. An additional drawback of the power doppler technique is that it suffers from motion artifact if used during portions of the cardiac cycle where the heart is moving.

An imaging technique that would allow good tissue suppression and signal to noise at power levels required to destroy microbubbles varies the amplitude and/or phase between pulses to suppress linear tissue signals. One possible technique was described in U.S. Pat. No. 5,577,505 but was not used in this manner. This patent describes a multi-pulse technique that involves changing amplitudes between transmit pulses and combining these pulses during receive to suppress the linear signals. With this technique as well as other multi-pulse techniques that have amplitude changes between transmit pulses and optionally phase changes as well, microbubbles exhibit a strong nonlinear response at the fundamental whereas tissue is suppressed since it behaves relatively linearly at the fundamental frequency. This tissue suppression at the fundamental frequency is opposed to purely harmonic based techniques (either pulse inversion or harmonic imaging) that have tissue harmonic signals present at low MI's (>0.1 or so)—often below the threshold required to destroy the microbubbles. There is also an improvement in signal to noise in operating at the fundamental frequency since attenuation is much lower than at the harmonic.

There are also multi-pulse techniques based on changing the amplitude and possibly phase between pulses and combining the pulses in such a manner that linear and/or non-linear tissue harmonic signals cancel (U.S. Pat. No. 6,361,498).

With these nonlinear detection techniques it is possible to image at MI's of 0.2 or higher—which are destructive for most contrast agents and to have minimal tissue signal—even when imaging at the harmonic. Frame rates of 25 Hz or lower allow enough time for some arteriole refill to occur.

As a means to further increase sensitivity and signal to noise of the arteriole contrast signal, the use of “coded” waveforms could be employed. Coded waveforms have been described in literature (e.g., U.S. Pat. No. 6,050,947) and involve transmitting a longer waveform to increase signal to noise. With proper “decoding” on receive the returning pulse can be compressed to gain back the loss of resolution. For example a “chirp” is a special case of a “coded” waveform and is a signal in which the frequency increases (‘up-chirp’) or decreases (‘down-chirp’) with time. These waveforms could be used in combination with the previous described multi-pulse detection techniques by modifying the amplitude and/or phase of the coded signal, decoding them on receive and combining them in a manner to suppress linear and/or non-linear signals.

Increased sensitivity can also be obtained by using an imaging sequence that uses an MI that is high enough to destroy contrast agent throughout the throughout the cardiac cycle (e.g., 0.2-0.8 depending on the microbubble characteristics) but then uses an even higher MI (e.g., 1.0) during systole—the portion of the cardiac cycle that has the blood in the arterioles “squeezed” into the imaging plane. This improves signal-to-noise by increasing the detection beamwidth to image more microbubbles as well as increasing the backscatter from each microbubble due to the higher power level. Other techniques could be used to get the same effect—such as increasing the beamwidth through focusing or apodization. A Matrix transducer allows for control of the elevation in this manner.

In order to make the results meaningful it is critical to calibrate the concentration of contrast. This can be accomplished by measuring the intensity in the myocardium throughout the cardiac cycle and normalizing to large intra-myocardial vessels that are typically seen during diastole (U.S. Pat. No. 6,730,036). In the scenario of the triggered imaging mentioned above this would require a 2^{nd} triggered frame during diastole to compare to. Another possible method would be to normalize to a large blood pool that represents 100% blood volume. This can be in the Left Ventricular cavity (U.S. Pat. No. 6,258,033) or a large vessel in the myocardium. For example, if the intensity in the myocardium during systole was 20 dB lower than the LV cavity and the concentration of microbubbles was still in the linear dosing range then myocardial arteriole blood volume would be 1%.

The invention described here is a method and apparatus for ultrasound imaging of microbubbles of a contrast agent in arterioles while virtually all microbubbles of the contrast agent have been eliminated in the capillaries of a patient and tissue signal response to ultrasound imaging is suppressed. This method and apparatus permits ultrasound imaging for detecting coronary artery disease without the need for a stress test.

The invention would be used to diagnose coronary artery disease without having a stress test. It could also serve as a quick screening tool for CAD.

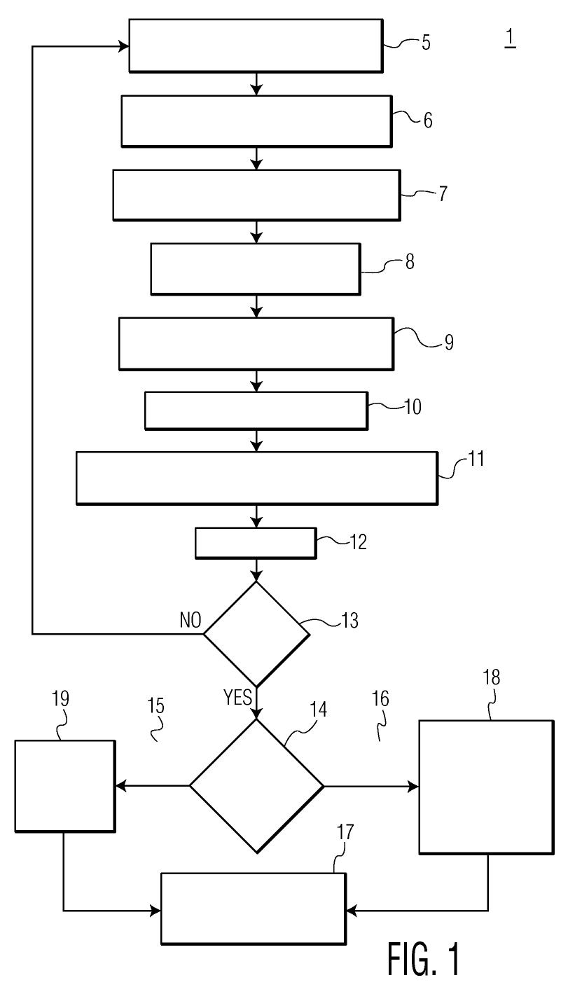

FIG. 1 is a flow chart illustrating a first technique for obtaining ultrasound images of microbubbles of a contrast agent in arterioles of a patient's body while eliminating or greatly reducing microbubbles in capillaries of the patient and tissue signal in accordance with the method and apparatus of the present invention;

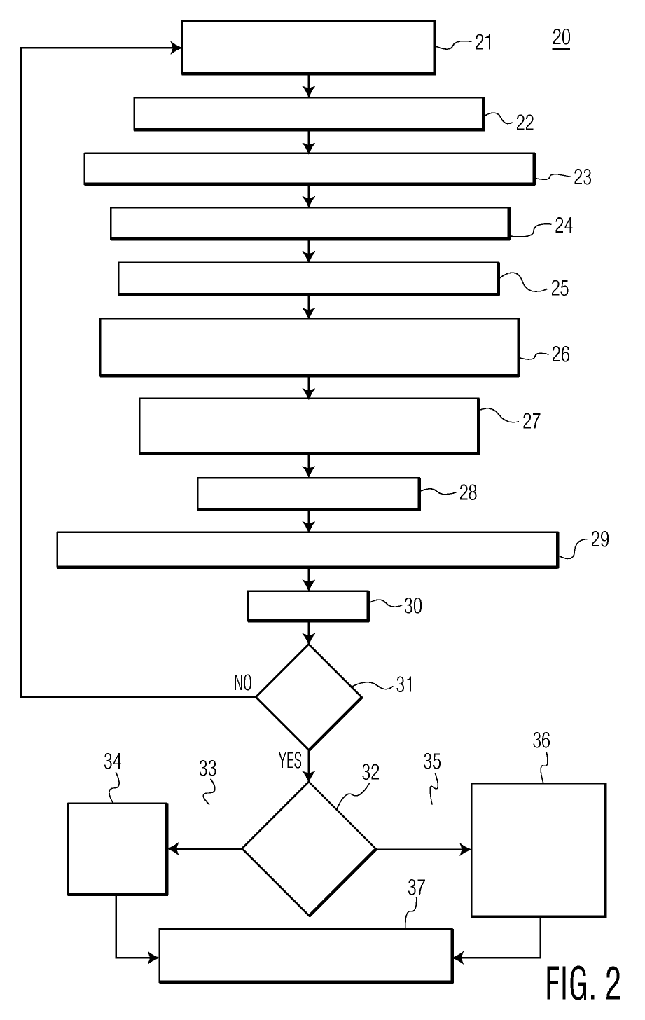

FIG. 2 is a flow chart illustrating a second technique for obtaining ultrasound images of microbubbles of a contrast agent in arterioles of a patient's body while eliminating or greatly reducing microbubbles in capillaries of the patient and tissue signal in accordance with the method and apparatus of the present invention; and

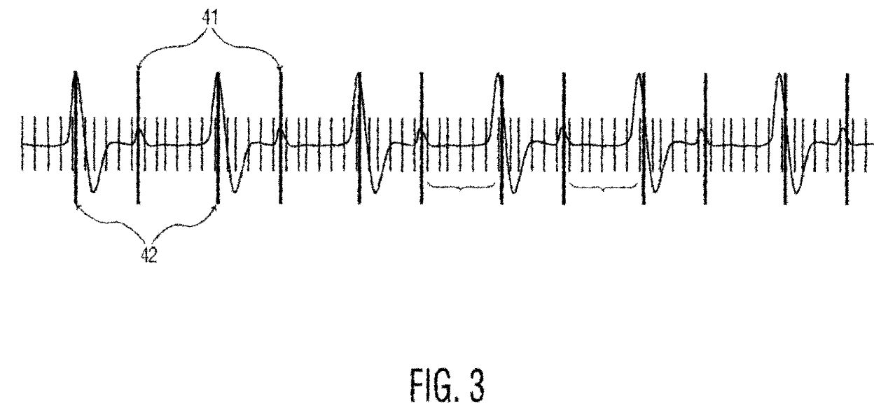

FIG. 3 is a diagram showing which portions of a cardiac cycle are imaged in a triggered mode and the rest of the cardiac cycle being imaged in non-triggered mode in accordance with the second technique of the present invention as shown in FIG. 2.

Referring now to drawings of FIGS. 1-3, FIG. 1 is a flow illustrating a first technique for imaging in accordance with the present invention.

As shown in FIG. 1, one first selects an imaging 3D subvolume or imaging plane 5 on the console of the ultrasonic imaging apparatus (such as a Philips 7500 Sonos) on a location .such as the apical location of a patient's body.

The present invention provides for imaging in subvolumes to include above and beyond a plane as a subvolume is more than one plane in an elevation dimension but could represent a smaller lateral dimension. Matrix transducers are capable of using subvolumes.

An imaging mode 6 is next selected for contrast destruction and tissue suppression. In the case of the first technique this can include setting a mechanical index for microbubble destruction 7 and setting a frame rate 8 to permit sufficient time for refilling larger vessels with contrast agent such as the arterioles. The mechanical index is preferably set to a value within a range of a range of 0.2 to 0.8. The frame rate is preferably set to a value within a range of 1 to 25 Hz.

In the case of the first technique by the use of a multi-pulse technique combining amplitude and phase modulation using the controls on the console of the ultrasound imaging apparatus, linear and optionally second order non-linear tissue signals are eliminated from the imaging by combining the pulses so that tissue noise is suppressed. Power and frame rates are chosen such that microbubble signals from the capillaries are eliminated.

In FIG. 1, other image settings on the ultrasound imaging apparatus are set, such as gain for best visualization of images 8. Contrast agent is then either injected or infused into a patient's body 10. When the contrast agent arrives, the gain, mechanical index, the frame rate, contrast delivery controls of the ultrasonic apparatus are set to optimal settings 11.

The ultrasound imaging apparatus 12 obtains images of the patient's body 13 and when all images are obtained 14, the images are calibrated or normalized, as described below for either the LV cavity 15 or the myocardial intensity and appropriate normalization for LV cavity intensity 19 or either the diastolic intensity or myocardial intensity 18 is obtained. Images or a graph of results are derived based on the normalized values 17.

The first technique of the present invention is different from that disclosed in U.S. Pat. No. 6,730,036 as the present invention discloses the use of fundamental detection techniques. U.S. Pat. No. 6,730,036 discloses the use of harmonic or ultraharmonic based techniques (filtering between harmonics). This first technique would use non-linear fundamental techniques including but not limited to those described in U.S. Pat. No. 5,577,505 and U.S. Pat. No. 6,361,498. These techniques suppress tissue very well in the mechanical index (MI) range that the present invention needs to image at (typically greater than 0.2 and less than 0.8) with the first technique of the present invention.

Calibration/normalization is necessary to assess the amount of contrast. This is true since there are many things that affect the intensity of a given frame. A higher contrast dose will give a higher intensity and a higher gain or higher power will give a higher intensity so in order to determine the concentration of contrast there must be something to compare the intensity of a given region of interest in a given frame to. In one case the intensity in the myocardium of end systolic frames can be compared to the intensity in the myocardium end diastolic frames. For example, the variation in the cardiac cycle could be 6 dB with end systole being 6 dB below end diastolic intensity. Alternatively the systolic/diastolic ratio (systolic intensity divided by diastolic intensity) could be generated. In the case of 6 dB the ratio of intensities would be 0.25. The other way to normalize is to compare locally to the LV cavity. Comparing locally is important (i.e. approximately same depth so acoustic parameters including MI and beam properties are as equal as possible in the tissue and in the cavity). Since the LV cavity is 100% blood the ratio of myocardial intensity to LV cavity will give an indication of the percent of blood (e.g., bubbles in the arterioles assuming we have isolated the arterioles by destruction of bubbles in capillaries).

The frame rate will control the time and therefore velocity of vessels that are being imaged. Velocities are higher in larger vessels so faster frame rates can also help isolate bigger coronary arteries as well as arterioles. Visualization of the larger vessels such as intramyocardial coronaries are primarily seen during diastole and help determine system settings such as imaging mode, Mechanical Index, Frame rate, and gain as well as contrast infusion rate. They also provide means for normalizing the systolic intensities.

FIGS. 2 and 3 describe the second technique of the present invention for a triggered mode scenario in which a portion of the patient's cardiac cycle is chosen, namely one trigger during systole and one during diastole, at which imaging is done by the ultrasound imaging apparatus at higher power with the rest of the cardiac cycle being imaged at a lower power. For a high power imaging the mechanical index is set to about to or greater than 0.5.

FIG. 3 shows the systolic and diastolic triggered frames utilizing technique 2 as described in the flow chart of FIG. 2. The chart is similar to that of FIG. 1 except this is for the triggered scenario or technique 2. Again, in FIG. 2 an imaging subvolume or plane is selected by the ultrasound imaging apparatus 21; the imaging mode is selected for microbubble destruction 22; the mechanical index 23 is set for equal to or greater than 0.2; the frame rate 24 is set for larger vessels, e.g. arterioles at less than or equal to 25 HZ. The image mode for detection of the contrast agent is then selected for the triggered images 25.

The imaging parameters are optimized for triggered images 26—settings such as delay from R-Wave, mechanical index, focusing, etc. The other settings such as gain are optimized for the best visualization of the images 27 and the steps 29-37 are similar to the steps in FIG. 1, namely, injecting or infusing the contrast agent into a patient 28; optimizing gain, mechanical index, frame rate, contrast delivery settings upon arrival of the contrast agent 29; acquiring the images 30; ascertaining that every view has been imaged 31; then proceeding with normalization 32 for either Left Ventricular (LV) cavity 33 or myocardial intensity 35 and in the case of LV cavity 33 normalizing to LV cavity intensity 34 and in the case of myocardial 35 normalizing 36 to either diastolic myocardial intensity or peak myocardial intensity and then deriving the image or graph of results base on these normalized values 37 for display on the screen of the ultrasonic imaging apparatus.

With this second technique, it is also possible that the detection technique and transmit and receive parameters are different in the triggered frames vs. the non-triggered frames. In this scenario the detection techniques include those mentioned in the first technique in FIG. 1, as well as techniques with filters set to receive energy in between harmonics (ultraharmonics) or harmonics as well as power Doppler techniques.

While presently preferred embodiments have been described for purposes of the disclosure, numerous changes in the arrangement of method steps and apparatus parts can be made by those skilled in the art. Such changes are encompassed within the spirit of the invention as defined by the appended claims.

1. An apparatus for detecting ultrasound contrast agents in arterioles of a patient's body, comprising:
an ultrasound imaging apparatus having a console panel with controls thereon, one of said controls being set to select an image mode for contrast detection and tissue suppression;
another of said controls setting a mechanical index a value that destroys microbubbles of a contrast agent in a patient;
another control for setting a frame rate to a value to permit for larger vessel refilling of contrast agent in arterioles of said patient;
other image controls being set to optimum settings to obtaining a best visualization of ultrasound images;
said contrast agent being either injected or infused into said patient;
at least one of a number of controls for gain, mechanical index, frame rate, contrast delivery, being set to an optimized setting, contrast delivery upon arrival of said contrast agent;
said ultrasound imaging apparatus receiving images of microbubbles of said contrast agent refilling into said arterioles of said patient;
said ultrasound imaging apparatus normalizing said received images; and
said ultrasound imaging apparatus having a display screen to display said normalized images to said ultrasound imaging apparatus as images or graphs.

2. The apparatus according to claim 1 further comprising setting said mechanical index to a value in a range of 0.2 to 0.8.

3. The apparatus according to claim 2 further comprising setting said frame rate to a value in a range of 1 to 25 Hz.

4. The apparatus according to claim 3 wherein by setting said mechanical index to said value within said range microbubbles are destroyed in said capillaries and said arterioles of said patient and a response for tissue signal to said ultrasound imaging is more linear compared with non-linear response by said microbubbles of said contrast agent refilling said arterioles during said short time interval set by said value of said range of said frame rate that is too short a time interval for said contrast agent to refill in said capillaries thereby permitting said ultrasound imaging apparatus to suppress said tissue signal and to image said microbubbles of said contrast agent in said arterioles of said patient.

5. The apparatus according to claim 1 further comprising:
said ultrasound imaging apparatus having controls set to vary an amplitude and phase between pulses of said tissue signal so that linear and/or non-linear harmonic signals of said tissue signal cancel each other out; and
said mechanical index is set to a level that eliminates said microbubbles in said capillaries.

6. The apparatus according to claim 1 wherein said ultrasound imaging apparatus utilizes a coded waveform technology wherein said ultrasound is transformed into a longer waveform during transmission to increase signal to noise ratio of said transmitted signal and upon returning is compressed back to recover loss of resolution.

7. An ultrasound imaging apparatus for detecting ultrasound contrast agents in arterioles of a patient's body, comprising:
said apparatus having a console with controls including controls for selecting an image mode for contrast destruction;
said control including a control for setting a mechanical index for destruction microbubble of a contrast agent in a patient;
said controls including a control for setting a frame rate of said ultrasound imaging apparatus for larger vessel refilling of contrast agent in arterioles of said patient;
said controls including a control for selecting imaging mode for triggered imaging frames for contrast detection and tissue suppression;
said controls including controls set to optimize image parameters for said triggered image frames independent from the destruction settings;
said contrast agent being either injected or infused into said patient;
at least one of a number of controls for gain, mechanical index, frame rate, contrast delivery, being set to an optimized setting, contrast delivery upon arrival of said contrast agent;
said ultrasound imaging apparatus receiving images of microbubbles of said contrast agent refilling into said arterioles of said patient;
said ultrasound imaging apparatus normalizing said received images; and
said ultrasound imaging apparatus having a display screen to display said normalized images to said ultrasound imaging apparatus as images or graphs.

8. The apparatus according to claim 7 further comprising said control set to optimize image portion of said triggered image frames including a multi pulse tissue suppression technique wherein said pulses have at least two different amplitudes and filters are set to image a fundamental frequency, said mechanical index of destruction imaging being set so that microbubble destruction occurs in a plane or sub volume of finite thickness and said frame rate of destruction imaging is set for a time between frames sufficient for arterioles to be refilled with said contrast agent but insufficient for capillaries to be refilled with said contrast agent.

9. The apparatus according to claim 8 where said mechanical index of destruction imaging is >=0.2.

10. The apparatus according to claim 8 where said mechanical index of triggered imaging is >=0.5

11. The apparatus according to claim 8 where said frame rate of destruction imaging is <=25 Hz.

12. The apparatus according to claim 8 wherein a signal in the myocardium is normalized to a large blood pool such as a left ventricular cavity or an intramyocardial vessel to obtain an accurate assessment of arteriole blood volume.

13. The apparatus according to claim 8 wherein intensity is measured in a given region of interest throughout the cardiac cycle.

14. The apparatus according to claim 8 wherein said imaging is synchronous to an Electrocardiogram (ECG).

15. The apparatus according to claim 8 wherein certain frames (the triggered frames) in a cardiac cycle of a patient use a higher power to increase a detection beamwidth.

16. The apparatus according to claim 8 wherein certain frames (the triggered frames) in a cardiac cycle of a patient have a different focusing or elevation beamwidth to increase a detection beamwidth.

17. The apparatus according to claim 8 wherein certain frames (the triggered frames) in a cardiac cycle of a patient have a different transmit waveform (frequency, pulse length) to increase a detection beamwidth.

18. The apparatus according to claim 7 wherein any one of various other detection techniques can be employed for the triggered images including ultraharmonics and power doppler (PD).

19. The apparatus according to claim 8 wherein the triggering at one point of time in diastole and one point of time in systole per cardiac cycle.

20. The apparatus according to claim 1 further comprising:
said ultrasound imaging apparatus utilizing a multi-pulse tissue suppression technique wherein at least 3 pulses are provided having different amplitudes in which a different amplitude can include either a different phase or peak amplitude combined so as to cancel linear and quadratic (i.e., harmonic) signals, wherein filters are set to image at least part of fundamental and harmonic frequencies, said Mechanical Index is set so that microbubble destruction occurs in a plane or subvolume of finite thickness, and said frame rate is set for a time between frames sufficient for arterioles to be refilled with said contrast agent but insufficient for capillaries to be refilled with said contrast agent.

21. The apparatus according to claim 7 wherein said ultrasound imaging apparatus utilizes a coded waveform technology wherein said ultrasound signal is transformed into a longer waveform during transmission to increase signal to noise ratio of said transmitted signal and upon returning is compressed back to recover loss of resolution.

22. The apparatus according to claim 21 wherein said ultrasound imaging apparatus utilizes a coded waveform technology wherein said ultrasound signal is transformed into a longer waveform during transmission to increase signal to noise ratio of said transmitted signal and upon returning is compressed back to recover loss of resolution wherein said coded waveform technique is used with said multi-pulse technique to modify said amplitude and/or phase of said coded waveform.

23. A method for detecting ultrasound contrast agents in arterioles of a patient's body, the steps comprising:
selecting an image mode for contrast detection and tissue suppression;
setting a mechanical index of an ultrasound apparatus for microbubbles of a contrast agent in a patient;
setting a frame rate of said ultrasound imaging apparatus for larger vessel refilling of contrast agent in arterioles of said patient;
optimizing other image settings of said ultrasound imaging apparatus for obtaining a best visualization of ultrasound images;
injecting or infusing said contrast agent in said patient;
optimizing at least one of a number of controls for gain, mechanical index, frame rate, contrast delivery upon arrival of said contrast agent;
acquiring images of microbubbles of said contrast agent refilling into said arterioles of said patient;
normalizing said acquired images; and
displaying said normalized images to said ultrasound imaging apparatus as images or graphs.

24. The method according to claim 23 further comprising the step of setting said mechanical index to a value in a range of 0.2 to 0.8.

25. The method according to claim 24 further comprising the step of setting said frame rate to a value in a range of 1 to 25 Hz.

26. The method according to claim 23 wherein by setting said mechanical index to said value within said range microbubbles are destroyed in said capillaries and said arterioles of said patient and a response for tissue signal to said ultrasound imaging is more linear compared with non-linear response by said microbubbles of said contrast agent refilling said arterioles during said short time interval set by said value of said range of said frame rate that is too short a time interval for said contrast agent to refill in said capillaries thereby permitting said ultrasound imaging apparatus to suppress said tissue signal and to image said microbubbles of said contrast agent in said arterioles of said patient.

27. The method according to claim 23 further comprising the steps of:
varying the amplitude and phase between pulses of said tissue signal so that linear and/or non-linear harmonic signals of said tissue signal cancel each other out; and
setting said mechanical index to a level that eliminates said microbubbles in said capillaries.

28. The method according to claim 23 wherein said imaging apparatus utilizes a coded waveform technology wherein said ultrasound signal is transformed into a longer waveform during transmission to increase signal to noise ratio of said transmitted signal and upon returning is compressed back to recover loss of resolution.

29. A method for detecting ultrasound contrast agents in arterioles of a patient's body, the steps comprising:
selecting an image mode for contrast destruction;
setting a mechanical index of an ultrasound apparatus for destruction microbubble of a contrast agent in a patient;
setting a frame rate of said ultrasound imaging apparatus for larger vessel refilling of contrast agent in arterioles of said patient;
selecting imaging mode for triggered imaging frames independent of first said destruction mode;
optimizing image parameters for said triggered image frames independent of first said destruction mode;
injecting or infusing said contrast agent in said patient;
optimizing at least one of a number of controls for gain, mechanical index, frame rate, contrast delivery upon arrival of said contrast agent;
acquiring images of microbubbles of said contrast agent refilling into said arterioles of said patient;
normalizing said acquired images; and
displaying said normalized images to said ultrasound imaging apparatus as images or graphs.

30. The method according to claim 29 further comprising setting said control set to optimize image portion of said triggered image frames including a multi pulse tissue suppression technique wherein said pulses have at least two different amplitudes and filters are set to image a fundamental frequency, said mechanical index of destruction imaging being set so that microbubble destruction occurs in a plane or sub volume of finite thickness and said frame rate is set for a time between frames sufficient for arterioles to be refilled with said contrast agent but insufficient for capillaries to be refilled with said contrast agent.

31. The method according to claim 29 where said mechanical index of destruction imaging is >=0.2.

32. The method according to claim 29 wherein said mechanical index of triggered imaging is >=0.5.

33. The method according to claim 29 wherein said frame rate of destruction imaging is <=25 Hz.

34. The method according to claim 29 wherein a signal in the myocardium is normalized to a large blood pool such as a left ventricular cavity or an intramyocardial vessel to obtain an accurate assessment of arteriole blood volume.

35. The method according to claim 29 wherein intensity is measured in a given region of interest throughout the cardiac cycle.

36. The method according to claim 29 wherein said imaging is synchronous to an Electrocardiogram (ECG).

37. The method according to claim 29 wherein said triggered frames in a cardiac cycle of a patient use a higher power to increase a detection beamwidth.

38. The method according to claim 29 wherein said triggered frames in a cardiac cycle of a patient have a different focusing or elevation beamwidth to increase a detection beamwidth.

39. The method according to claim 29 wherein said triggered frames in a cardiac cycle of a patient have a different transmit waveform (frequency, pulse length) to increase a detection beamwidth.

40. The method according to claim 29 wherein any one of various other detection techniques can be employed for the triggered images including ultraharmonics and power doppler (PD).

41. The method according to claim 29 wherein the triggering is at only one point of time in diastole and at one point of time in systole per cardiac cycle.

42. The method according to claim 29 wherein said ultrasound imaging apparatus utilizes a multi-pulse tissue suppression technique wherein at least 3 pulses are provided having different amplitudes in which a different amplitude can include either a different phase or peak amplitude combined so as to cancel linear and quadratic (i.e., harmonic) signals, wherein filters are set to image at least part of fundamental and harmonic frequencies, said Mechanical Index is set so that microbubble destruction occurs in a plane or subvolume of finite thickness, and said frame rate is set for a time between frames sufficient for arterioles to be refilled with said contrast agent but insufficient for capillaries to be refilled with said contrast agent.

43. The method according to claim 29 wherein said ultrasound imaging apparatus utilizes a coded waveform technology wherein said ultrasound signal is transformed into a longer waveform during transmission to increase signal to noise ratio of said transmitted signal and upon returning is compressed back to recover loss of resolution.

44. The method according to claim 29 wherein said ultrasound imaging apparatus utilizes a coded waveform technology wherein said ultrasound signal is transformed into a longer waveform during transmission to increase signal to noise ratio of said transmitted signal and upon returning is compressed back to recover loss of resolution wherein said coded waveform technique is used with said multi-pulse technique to modify said amplitude and/or phase of said coded waveform.
